(19) United States
(12) Plant Patent Application Publication
(10) Pub. No.: US 2008/0235834 P1
(43) Pub. Date: Sep. 25, 2008

(54) Rhododendron Rootstock named "Rhodunter 149"

(75) Inventor: Walter Preil, Ammersbek (DE)

Correspondence Address: THE WEBB LAW FIRM, P.C., 700 KOPPERS BUILDING, 436 SEVENTH AVENUE, PITTSBURGH, PA, 15219, US

(73) Assignee: Inkarho GmbH, Bad Zwischenahn (DE)

(21) Appl. No.: 12/070,196

(22) Filed: Feb. 15, 2008

(30) Foreign Application Priority Data
Mar. 19, 2007 (EP) 2007/0667

(51) Int. Cl. A01H 5/00

(52) U.S. Cl. PLT/238

(57) ABSTRACT

A new and distinct Rhododendron hybrid plant used as a rootstock that exhibits a high lime tolerance and vigorous growth.

### BOTANICAL CLASSIFICATION

Rhododendron hybrid

### VARIETAL DENOMINATION

‘Rhodunter 149’

### BACKGROUND OF THE INVENTION

The present invention comprises a new and distinct cultivar of Rhododendron hybrid used as a rootstock known by the varietal name ‘Rhodunter 149’. The new variety was discovered in Ahrensburg, Germany. The new variety is the result of a planned breeding program between an unpatented Rhododendron fortunei (female parent) and C. V. ‘Cunningham's White’ (unpatented male parent). ‘Rhodunter 149’ exhibits leaves and flowers that are more similar to its female parent than to its male parent. Additionally, ‘Rhodunter 149’ exhibits more vigorous growth when compared to its male parent. Further, the new variety differs from both parents in its improved tolerance to lime. In fact, such improved lime tolerance (no Fe-deficiency symptoms on soils up to 6.9 pH) distinguishes the new variety from other Rhododendron varieties known to the breeder. ‘Rhodunter 149’ has been trial and field tested and has been found to retain its distinctive characteristics and remain true to type through successive propagations.

### DESCRIPTION OF THE DRAWINGS

The accompanying photographic drawings illustrate the new variety, with the color being as nearly true as is possible with color illustrations of this type.

FIG. 1 shows a view of a typical flowering plant of the new variety grown to a flowering plant; and

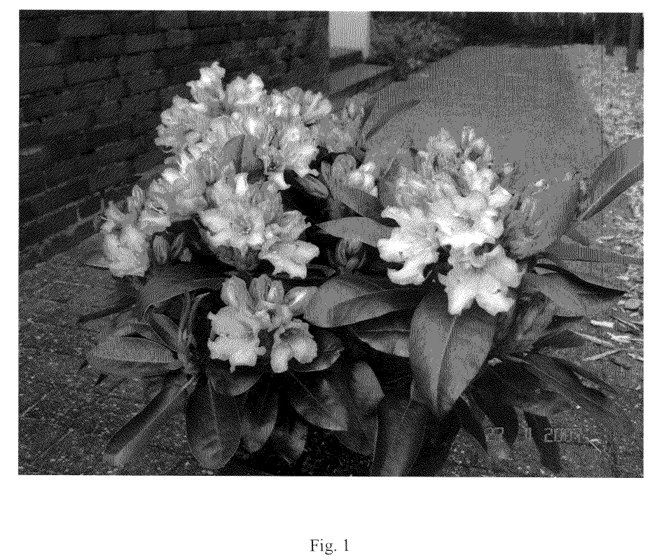

FIG. 2 shows a close-up view of the inflorescences of the new variety.

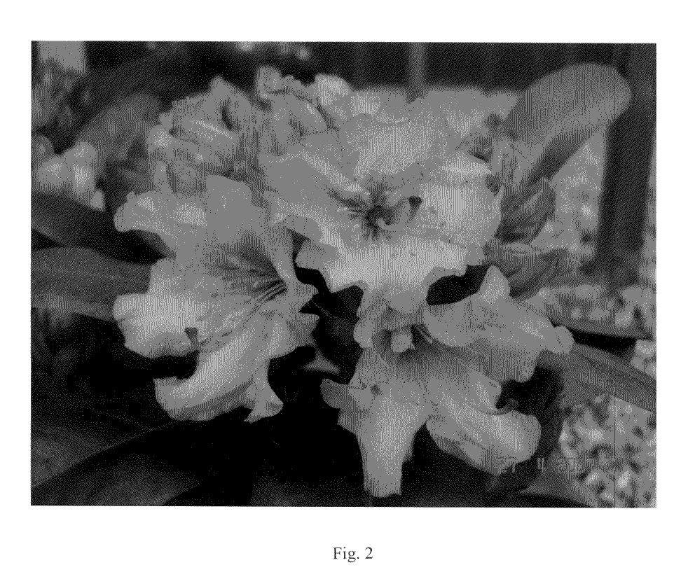

### DESCRIPTION OF THE PLANT

The following detailed description sets forth the characteristics of the new cultivar. The data which defines these characteristics were collected by asexual reproductions performed via cuttings or in vitro culture carried out in Ahrensburg, Germany. The color readings were taken in natural daylight. Color measurements were taken outside under natural daylight. Color references are primarily to the R.H.S. Colour Chart of The Royal Horticultural Society of London.
Plant:
   Time to initiate roots.—About 90 days at 22-25° C.
   Time to produce a finished flowering plant from a rooted cutting.—About 4 years in the field.
   Rooting habit.—Compact root bales.
   Form.—Spreading.
   Height.—50 cm at 3 years.
   Diameter.—40 cm at 3 years.
   Vigor.—Strong.
   Branching habit.—Compact.
   Disease, drought and pest resistance.—No observations made to date.
   Trunk (at 4 years).—Diameter: 3 cm. Height: 5 cm above soil. Bark color: Greenish-red/brown. Texture: Hard. Pubescence: Absent. Form: Erect.
   Lateral branches.—Diameter: 5-7 mm. Internode length: 1-2 cm. Texture: Hard. Pubescence: Absent. Color: Greenish-red/brown.
   Leaves.—Arrangement: Alternate. Length: 20 cm maximum. Width: 7.5 cm maximum. Shape: Oblanceolate. Apex: Broad. Base: Tapering. Pubescence: Absent. Color: Upper surface: 147B for young leaves and 147A for mature leaves. Lower surface: 147C for both young and mature leaves. Margins: Entire. Venation pattern: Normal Rhododendron leaf pattern. Vein color: Upper surface: 144A. Lower surface: 145C. Bracts: None present.
   Flower.—Natural flowering season: Beginning of May until the end of May in Northern Germany. Fragrance: Typical of Rhododendron fortunei. Buds (described before bursting): Shape: Oblong. Width: 20 mm. Length: 40 mm. Color: 67A. Spike: Number of flowers per spike: 10-15. Length: 15 cm. Diameter: 15 cm. Corolla petals: Shape: Widely funnel-shaped. Number: 7. Length: 45-55 mm. Width: 80 mm. Apex: Round. Margin: Undulate. Color: When opening: 67B. Fully opened (upper surface): 74C to 74D. (lower surface): 84B/C. Corolla throat: Tube length: 20 mm. Color: 153A. Texture: Wax-like.
   Reproductive organs.—Stamens: Number: 14. Length: 30 mm. Anthers: Shape: Normal Rhododendron type. Color: Yellow. Length: 25 mm. Pollen: Amount of pollen: Medium. Color of pollen: Light brown. Pistil: Number: 1. Style: Color: Yellow. Length: 45 mm; filiform. Stigma: Shape: Undivided. Color: Brown.
   Seeds.—Production: Seed capsules mature in November when pollinated in May. Length: 2 mm. Color: Brown. Number: Approximately 200 per capsule.

I claim:

1. A new and distinct variety of Rhododendron hybrid plant, as is herein illustrated and described.
